(19) United States
(12) Patent Application Publication
(10) Pub. No.: US 2002/0076429 A1
(43) Pub. Date: Jun. 20, 2002

(54) BONE PASTE SUBJECTED TO IRRADIATIVE AND THERMAL TREATMENT

(76) Inventors: JOHN F. WIRONEN, GAINESVILLE, FL (US); PHILIP A. FELTON, ALACHUA, FL (US); REBECCA JAW, ALACHUA, FL (US)

Correspondence Address: VAN DYKE & ASSOCIATES, P.A., 1630 HILLCREST STREET, ORLANDO, FL, 32803, US

(21) Appl. No.: 09/154,400

(22) Filed: Sep. 16, 1998

Related U.S. Application Data
(63) Continuation-in-part of application No. 09/014,519, filed on Jan. 28, 1998.

(51) Int. Cl. A61K 9/00; A61K 6/00; A61K 7/00

(52) U.S. Cl. 424/426

(57) ABSTRACT

A thermally sterilized bone paste useful in the orthopedic arts, for example in the repair of non-union fractures, periodontal ridge augmentation, craniofacial surgery, implant fixation, impaction grafting, or any other procedure in which generation of new bone is deemed necessary, is provided by a composition comprising a substantially bioabsorbable osteogenic compound in a matrix of 11-19%, and preferably about 15-19% (w/w) or thermally sterilized gelatin. In various embodiments, the osteogenic compound is selected from (i) demineralized bone matrix (DBM); (ii) bioactive glass ceramic, BIOGLASS®, bioactive ceramic, calcium phosphate ceramic, hydroxyapatite, hydroxyapatite carbonate, corraline hydroxyapatite, calcined bone, tricalcium phosphate, or like material; bone marrow extracts, vascular proliferation or regeneration growth factors, bone morphogenetic protein, TGF-β, PDGF, or mixtures thereof, natural or recombinant; and (iv) mixtures of (i)-(iii). The thermally sterilized gelatin may be a commercially available grade of gelatin which is both thermally and irradiatively sterilized.

### CROSS REFERENCE TO RELATED APPLICATIONS

This application is a continuation-in-part of related U.S. patent application Ser. No. 09/014,519, filed Jan. 28, 1998, pending

### BACKGROUND OF THE INVENTION

1. Field of the Invention

This invention provides an improved, thermally sterilized bone paste, useful in the production of osteogenic, osteoinductive, and/or osteoconductive compositions for use in the field of orthopedic medicine to achieve bone fusions, fusion of implants to bone, filling of bone defects, or any other applications in which an osteoinductive, osteogenic composition is desirable.

2. Background

More than 100,000 bone grafting procedures are performed every year in the United States alone. (Cornell). In the majority of reconstruction procedures, the graft material is used as a filler between bone particles in the belief that continuous contact between particles of bone leads to more rapid and complete healing at the repair site (as well as greater mechanical integrity). (Bloebaum). In the cases of bone augmentation and spinal fusion, these bone grafts may make up the entire structure of the graft, since there are no bone fragments in the area. With the possible exception of one product (whose use guidelines do not allow this), all bone grafting materials require surgical placement with the requisite incisions.

Osteogenic bone grafting materials may be separated into two classes, namely those which are osteoconductive, and those which are osteoinductive. While the exact definition of these terms remains a matter of debate, it can be said that osteoconductive implants “conduct” bone growth across defects when implanted into osseous tissue. (Einhorn). Osteoinductive implants, on the other hand, have the ability to “induce” cells in the area to generate bone of their own accord. (Einhorn). These osteoinductive implants will cause the generation of bone even when they are implanted into non-osseous tissue (e.g. subcutaneous or intramuscular implantation). (Einhorn; Benedict; Strates; Urist).

All of the artificially produced bone-grafting materials available today fall in the osteoconductive category of grafts. Among these are Bioglass®, Norian®, Collagraft®, corraline hydroxyapatite, powdered hydroxyapatite, crystalline and amorphous hydroxyapatite (hydroxyl apatite), and a number of other products. All of these implants rely on their similarity to natural bone hydroxyapatite. A likely mechanism for bone conduction lies in the ability of these materials to enhance diffusion of trophic factors and cells over their very large surface areas and the mechanical support which they provide to growing tissues. FIG. 1 provides a list of relevant properties of selected bone graft materials.

The other category of bone grafting materials currently available is encompassed by autograft or allograft bone. If not too harshly processed, these materials are generally osteoinductive.(Yazdi). Since they are tissue transplants, their use imposes certain risks. Autografts have been associated with harvest site morbidity in excess of 20%. (Younger). Frozen or freeze-dried allografts induce some immune response, and if not properly screened, can be associated with disease transmission. (Hordin). The last variety of allografts is demineralized bone matrix.

Demineralized Bone Matrix (DBM) was first described by Senn in 1889. (Senn). It was rediscovered, largely by accident, and thoroughly studied by Urist and Strates in the late 1960's. (Strates; Urist). It has since become a major product of tissue banks around the world. As the name implies, it is bone which has been demineralized by treatment with acid. A detailed outline of the process for producing this product is provided in FIG. 2.

DBM has the ability to induce the formation of bone even in non-osseous tissues within 4 weeks. (Strates; Urist; Lasa). The standard technique for determining the activity of DBM is to implant it subcutaneously or intramuscularly. (Nathan). It is believed that the major active factor in DBM is one or more bone morphogenetic proteins (BMP), (see U.S. Pat. No. 4,294,753, herein incorporated by reference). Other growth factors, including but not limited to TGF-beta, (see U.S. Pat. No. 5,422,340, herein incorporated by reference), platelet derived growth factor (PDGF), and the like, may be important for this function also.

Bioglass® is a bone grafting material which is a SiO₂, Na₂O, CaO, P₂O₅ glass which has the ability to produce a bio-active surface layer of hydroxyapatite carbonate within minutes of implantation. (Hench).

Two problems are associated with the use of DBM or Bioglass. Both of these materials are supplied as large particles, and do not always stay in the area into which they are implanted. (Scarborough; Frenkel). Also, due to their coarse nature, they are hard to mold and handle in the operating room. Accordingly, there is the need for a product which does not allow for particle migration, while also being easier to use in the operating environment.

As noted in table 1, in recent years, several bone-filling surgical pastes have become commercially available. These products range from simple mixtures of saline with a sand-like powder to a recently released gel, known as GRAFTON®, a glycerol-based, non-cross-linkable composition. All of these products are used in orthopedics to repair bone defects, such as voids, cavities, cracks etc. Such defects may be the result of trauma or may be congenital, and the known pastes may be used to patch or fill such defects, or build upon existing bony structures. The ultimate goal of such treatments is that the paste will induce bone formation to replace the paste while retaining the form created by the surgeon when applying the paste.

Desirably, a bone paste would be osteoconductive (i.e. it conducts bone cells into a region) and osteoinductive (i.e. stem cells are induced to differentiate into bone forming cells which begin production of new bone). In general, bone pastes known in the art are osteoconductive, with only weak osteoinductive effects. Accordingly, such known pastes are inadequate for filling of large voids and frequently do not effect proper bone formation even in small voids. All currently available bone pastes, including those that exhibit some osteoinductive activity, are difficult to handle, do not adequately remain at the site of implantation, or both.

Thus, one commercially available product, GRAFTON®, (see U.S. Pat. No. 5,484,601) is a non-cross-linkable composition of demineralized bone powder suspended in a polyhydroxy compound (e.g. glycerol) or esters thereof, optionally including various other ingredients, including gelatin. It is considered likely that this material is rapidly washed away from the implant location as the carrier matrix is glycerol, which is water soluble.

U.S. Pat. Nos. 5,236,456 and 5,405,390 (O'Leary and Prewett) outline an “osteogenic” gel composition which is made from demineralized bone matrix (DBM) by treating with concentrated acid (3 M HCl) and heating to between 40 and 50° C. The patent briefly describes mixing the gel with DBM and several other components. However, the method of manufacturing the gel composition is such that it produces mostly collagen fibers (i.e. the temperature elevation is insufficient to produce gelatin). As a result, the collagen fibers are not soluble in neutral solutions. To obtain a gel, the patent specifies that the collagen must be dissolved in acid of low pH (e.g. HCl or 1% acetic acid, at a pH of less than 4.0). However, compositions of low pH are not typically very compatible with biological implantations. It is also noted that at column 5, line 20, and column 6, line 15, it is specified that the temperature at which the gel solidifies is 0-5° C., which precludes gellation in vivo.

U.S. Pat. No. 4,440,750 (Glowacki and Pharris) outlines a standard enzymatic technique for extracting collagen from tissue using Pepsin. A highly refined collagen is obtained from animal sources, which is then reconstituted prior to forming the working composition. The collagen will not readily cross-link without the addition of other chemicals (e.g. aldehydes, chondroitin sulfate), which they do not specify in the composition. There is no mention of a set temperature or any reference to cross-linking behavior.

In U.S. Pat. Nos. 4,394,370 and 4,472,840, (Jefferies), complexes of reconstituted collagen with demineralized bone or solubilized bone morphogenetic protein, optionally cross-linked with glutaraldehyde, were reported to be osteogenic when implanted in vivo. The reconstituted collagen of these patents is pulverized, lyophilized, microcrystalline collagen which has been dialyzed to remove the hydrochloric acid used in collagen preparation. Accordingly, the composition of those patents does not involve the conversion of collagen to gelatin prior to formation of the composition. Hence, the composition would not exhibit the thermal cross-linking behavior of the instant composition.

In U.S. Pat. No. 4,678,470 (Nashef et al.) disclosed a non-resorbable bone-grafting material comprising demineralized bone matrix that had been cross-linked by treatment with glutaraldehyde, or like cross-linking agent, suspended in a gelatinous or semi-solid carrier. Given that the demineralized bone of that patent is chemically cross-linked, its bone inductive properties are considered to be destroyed and the composition essentially forms a structural filler or matrix into which recipient bone may grow.

In WO 89/04646 (Jefferies), a bone repair material having good structural strength was disclosed. The material comprised a demineralized bone matrix which had been surface activated by treatment with glutaraldehyde or like cross-linking agent to increase the binding thereof to biocompatible matrices. The resulting material has such a rigid structure that, prior to implantation into a biological recipient, the material may be machined.

In U.S. patent application Ser. No. 08/816,079, a bone paste comprising gelatin and demineralized bone matrix was disclosed. In that disclosure, the gelatin carrier matrix was not subjected to dehydrothermal cross-linking, as is the case for the instant ateliopeptide collagen carrier matrix.

The thermally sterilized bone paste of the present invention meets the needs in the art by providing a new material that is easy to handle and store, which adheres to the site of implantation, depending on the specific embodiment used, displays both osteoconductive and osteoinductive activities, it thermally cross-links at a concentration of between about fifteen to about nineteen weight percent at 38° C., as compared to the “Bone Paste” of U.S. application Ser. No. 08/816,079, which thermally cross-linked at between about twenty and forty-five weight percent, and is substantially bioabsorbable. Further, as compared with the “Thermally Sterilized Bone Paste” of U.S. application Ser. No. 09/014,519, the thermally sterilized bone paste of this invention is produced by a novel process including gamma irradiation, autoclaving, and blending with constituents by a novel process that permits commercial grades of gelatin to be processed for human or animal implantation.

Preferably, the composition of this invention is provided as a gel which contains mineral and protein components which have been clinically shown to induce rapid bone ingrowth. The composition may be delivered to the surgeon in a pre-loaded syringe, ready for use. Preferably, at a first temperature, the gel is easily formable into any shape, and is adhesive. Once inside the biological milieu, or at a second lower temperature, the gel desirably hardens as a rubbery solid, which does not wash away or migrate from the site of implantation. Upon ingrowth of bone, the implant material becomes completely incorporated into the biological system. The mode of making and using this composition is set forth in detail below.

### BRIEF SUMMARY OF THE INVENTION

A thermally sterilized bone paste useful in the orthopaedic arts, for example in the repair of non-union fractures, periodontal ridge augmentation, craniofacial surgery, implant fixation, arthrodesis of spinal or other joints, including spinal fusion procedures, or any other procedure in which generation of new bone is deemed necessary, is provided by a composition comprising gelatin and additional osteogenic components. The gelatin is preferably thermally cross-linked at about 38° C., at a gelatin concentration of between about 11-30%, and preferably at between about 15%-19% (w/w), and the osteogenic components are selected from:

(i) demineralized bone, preferably derived from the species into which the thermally sterilized bone paste is to be implanted; or

(ii) bioactive glass ceramic, BIOGLASS®, bioactive ceramic, calcium phosphate ceramic, hydroxyapatite, hydroxyapatite carbonate, corraline hydroxyapatite, calcined bone, cortical bone chips, cancellous bone chips, tricalcium phosphate, like material, or mixtures thereof; or

(iii) bone morphogenetic protein, osteogenic proteins or peptides (e.g. osteogenin, p15, CDMP, and the like), TGF-beta, bone marrow extracts, vascular proliferation or regeneration growth factors, PDGF, or mixtures thereof, natural or recombinant; or

(iv) mixtures of (i)-(iii).

Where present (ii) or like material is included to enhance the range of manipulable characteristics of strength and osteoinduction exhibited by the composition, and may comprise between about 0-60%, including about 40%, of the mass on a weight basis of the composition. Where present, (iii) reduces the need for demineralized bone, which otherwise provides a source of osteoinductive factors.

Demineralized bone has been shown to be highly effective in inducing bone formation. The gelatin provides a cross-linkable, adhesive and easily manipulated matrix in which the osteoconductive and osteoinductive elements of the composition are carried. Other factors, such as antibiotics, bone morphogenetic or other proteins, whether derived from natural or recombinant sources, wetting agents, glycerol, dextran, carboxymethyl cellulose (CMC), growth factors, steroids, non-steroidal anti-inflammatory compounds, or combinations thereof or any other material found to add to the desirable properties of the essential composition of this invention may be included.

The composition may be freeze-dried or pre-constituted, and may be provided in a convenient dispensing device, such as a pre-loaded syringe. The gel is preferably in a liquid or highly malleable state at temperatures above about 400° C., but sets up as a hard gel at or preferably slightly above the body temperature of the organism into which it is implanted (e.g. at 38° C. in humans).

### BRIEF SUMMARY OF THE FIGURES

FIG. 1 is a chart of existing bone grafting materials.

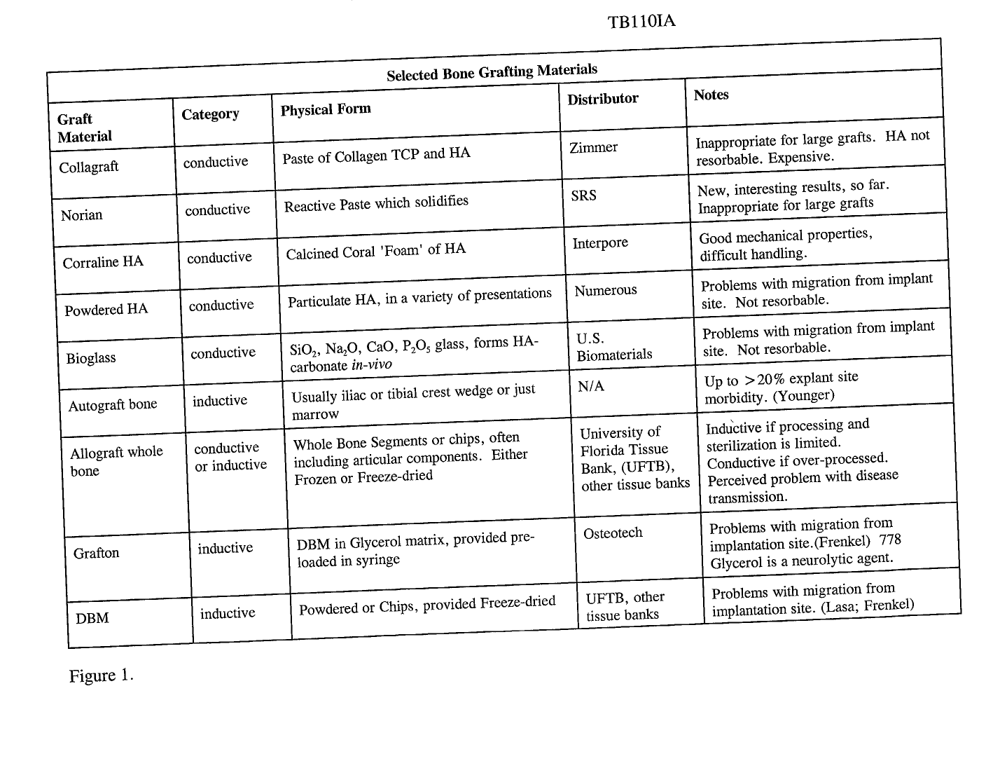

FIG. 2 represents a bone demineralization process.

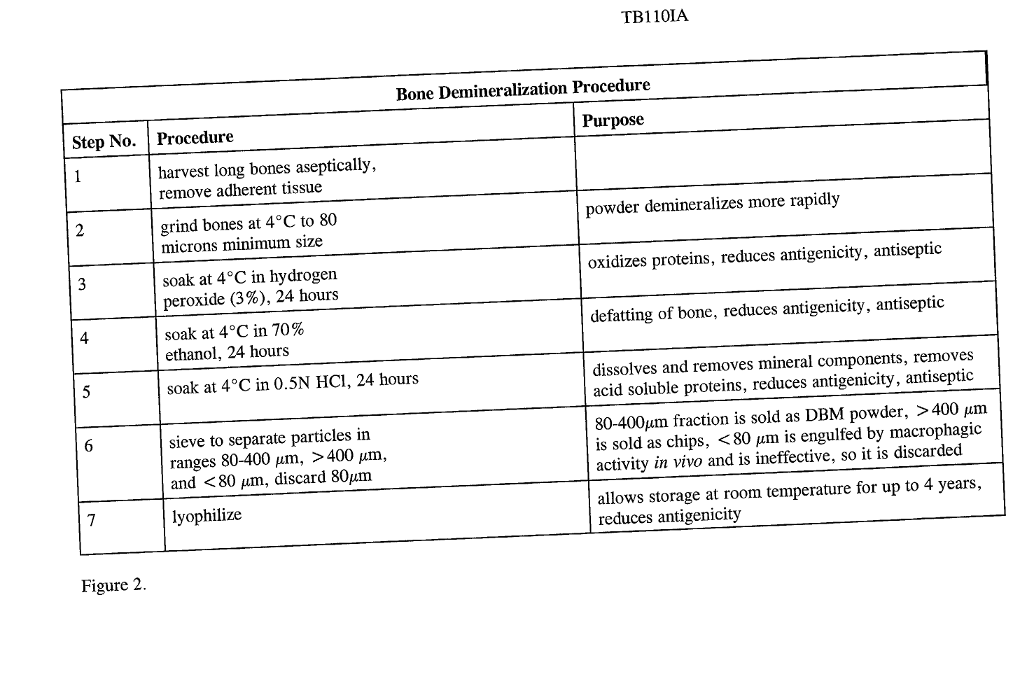

FIG. 3 is a graph of the kinematic viscosity (centistokes) versus concentration (%) for gelatin thermally sterilized or not thermally sterilized in a dry state, followed by dissolution in water and measurement of the kinematic viscosity.

### DETAILED DESCRIPTION OF THE INVENTION

It will be appreciated by those skilled in the art that the specifics of the composition of this invention, its method of preparation and use are applicable to such compositions for use in any vertebrate species. Nonetheless, because human use is considered likely to be the principal orthopedic application of this new material, the following description concentrates on exemplifying this material for human applications.

The composition of this invention comprises gelatin and additional osteogenic components. The gelatin is preferably thermally cross-linked at about 380° C., at a gelatin concentration of between about 11-30%, and preferably at between about 15% -19% (w/w), and the osteogenic components are selected from:

(i) demineralized bone, preferably derived from the species into which the thermally sterilized bone paste is to be implanted; or

(ii) bioactive glass ceramic, BIOGLASS®, bioactive ceramic, calcium phosphate ceramic, hydroxyapatite, hydroxyapatite carbonate, corraline hydroxyapatite, calcined bone, cortical bone chips, cancellous bone chips, tricalcium phosphate, like material, or mixtures thereof; or

(iii) bone morphogenetic protein, osteogenic proteins or peptides (e.g. osteogenin, p15, CDMP, and the like), TGF-beta, bone marrow extracts, vascular proliferation or regeneration growth factors, PDGF, or mixtures thereof, natural or recombinant; or

(iv) mixtures of (i)-(iii).

The composition is fluid at a first temperature (e.g., above 38° C.) and becomes thermally cross-linked at or just above a second temperature, corresponding to the normal body temperature of the organism into which the composition is to be implanted (e.g., at 38° C. in humans).

The terms “thermally cross-linked” or “thermally cross-linkable” are used herein to describe the property of a composition which contains molecules which, at or below a given temperature and concentration, associate in such a fashion as to result in gellation of a solution containing these molecules.

The term “thermally sterilized” is used herein to indicate that a material has been treated under such conditions of temperature as are generally recognized in the art to render a material sterile (i.e. devoid of living organisms). For example, the standard procedure of “autoclaving” a material occurs in a sealed chamber into which steam is pumped to such a pressure that the temperature within the chamber reaches approximately 121° C. Treatment of twenty minutes under such conditions is generally recognized as being sufficient to surface sterilize an object, with longer periods being required, depending on the volume of an object or liquid, through which heat is to be transferred. Other conditions of dry heat (i.e. absent steam) are also generally acknowledged as producing a sterile environment, as in, for example, approximately 121-130° C. of dry heat for from about five minutes to about six hours. Once again, considerations of time of exposure are required in order to achieve a sterile field. In any event, this term as applied to the composition of this invention does not require that the material described as “thermally sterilized” remain in a sterile state. In other words, the material may be implanted, in which case it would preferably remain sterile, or it may be left exposed on a shelf in an open and contaminated state, and yet still have been “thermally sterilized”. It is the physical characteristics of the thus-treated material (i.e. molecular weight and solution behavior, as revealed by the kinematic viscosity), that is critical, rather than the state of being sterile or not. According to this disclosure, treatment of a dried gelatin composition for approximately 5 minutes to about 18 hours, and preferably between about 3-6 hours at between about 121° C. to 130° C. is considered to come within the meaning of the term “thermally sterilized”.

The term “substantially bioabsorbable” is used herein to describe the property of a material which is no longer detectable at the site of implantation or has been remodeled at that site to create endogenous tissue after a reasonable period of biological resorption, such as three months to a year later. Accordingly, for example, demineralized bone matrix which has been chemically cross-linked with an agent such as glutaraldehyde, is not considered to be substantially bioabsorbable. However, demineralized bone matrix itself, gelatin, and bone morphogenetic factors are all considered to be substantially bioabsorbable as they cooperate in new bone formation, rather than purely providing structural rigidity or support, without being remodeled into new, endogenous tissue.

The gelatin acts as a carrier phase and has the ability to thermally cross-link over a very small temperature range. This thermal cross-linking reaction is largely controlled by physical entanglement and hydrogen bonding between chains, and so is dependant on concentration and temperature. (Sperling). Additionally, since gelatin has been used extensively in the medical market, its in vivo properties are thoroughly studied. (McDonald). The gel-foam sponge is the most familiar application of this biopolymer. Studies have indicated that gelatin is only mildly antigenic upon implantation, and is comparable in some of its properties to collagen, (McDonald). However, collagen does not exhibit the thermal cross-linking property so important to the composition of this invention.

Where present, the bioactive glass, such as BIOGLASS®, bioactive ceramic, calcium phosphate ceramic, hydroxyapatite, hydroxyapatite carbonate, calcined bone, tricalcium phosphate, or like material, is included to enhance the range of manipulable characteristics of strength and osteogenesis (osteoinduction and osteoconduction) exhibited by the composition.

The manufacture of gelatin is based on the partial hydrolysis of collagen. Collagen is available from skin, bone, cartilage, tendon and other connective tissue. Skin and bone yield Type I and Type III collagen molecules, while tendon yields nearly pure Type I collagen, and cartilage yields a mixture of Type II and rarer types of collagen molecules. Gelatin molecules resemble collagen triple helices, however, they are partially hydrolyzed. As a result, in solution they have little organization. But, as the solution cools, the gelatin molecules begin to form helical structures. As the solution cools further, the viscosity increases and a phase transformation from a solution to a gel occurs. This phase change is reversible when heat is added.

The set time and set temperature of a gelatin solution are dependent on the concentration of gelatin in solution, the molecular weight, or intrinsic viscosity, of the gelatin molecules, and the pH of the solution. At the isoelectric point, or the pH at which the gelatin molecules are electrically neutral, the set time is the shortest.

Collagen can be partially hydrolyzed by several methods. The Type A process is the simplest and most rapid process, in which dilute acid (e.g. less than 1 M HCl) is used to partially hydrolyze the collagen. Type A processing is generally used with porcine skin and demineralized bovine bone. The Type B process uses an alkaline solution to partially hydrolyze the collagen. Type B processing is generally used with bovine hide and demineralized bovine bone. Finally, enzymes, such as pepsin, may be used to partially hydrolyze collagen. Pepsin preferentially cleaves peptide bonds between aromatic amino acids. In collagen, treatment with pepsin converts native collagen, which contains telopeptide, to atelopeptide material, which reduces the level of interchain disulfide bonding that can occur in the collagen tertiary structure.

As one example of this method, the gelatin is prepared from the bones of the species into which the compositions are to be implanted, by crushing and defatting the bones followed by demineralization in 0.5 N HCl and then soaking for about 24 hours in approximately 300 mg/L pepsin in a 0.5 M acetic acid at 33° C. We have discovered that the yield of gelatin is enhanced by conducting two such extractions, one at about 30° C., and a second at about 33° C., and then pooling the product. The pH of the resulting solution is brought to about 7.0 with sodium hydroxide to denature the pepsin. The temperature of the solution is raised to between about 60-65° C. for about 12 to 30 minutes and returned to 19-200° C., or like temperature at which the gelatin remains soluble, to effect denaturation of remaining collagen and complete conversion to gelatin. The resulting solution is filtered to remove particulates and dialyzed or diafiltered against distilled water for 48 hours in a 30K-100K molecular weight cut-off (30K-100K MWCO) dialysis or diafiltration membrane.

In one embodiment of this invention, the gelatin thus produced is next lyophilized, preferably in sealable vials. The vials are filled (i.e. the vacuum is replaced) with a dry, inert gas, such as nitrogen, argon or the like, sealed, and then autoclaved (or otherwise heated, for example in a dry oven, to about 121-130° C.), in the sealed environment. This treatment has been found to increase the kinematic viscosity of the gelatin upon subsequent solubilization, permitting a lower effective concentration of the gelatin to be used in the bone paste to achieve gellation at about thirty-eight degrees centigrade, than has heretofore been possible. Depending on the level of moisture permitted to remain in the sealed environment, the level of increase in kinematic viscosity, measured subsequently, can be controlled in a dose-dependent fashion (the increase in viscosity is quenched by humidity). After lyophilization and thermal treatment, the gelatin is redissolved in phosphate buffered saline (PBS) or water to a sufficiently high effective concentration such that a final composition comprising from about 11-19%, and preferably about 15-19% (w/w) gelatin may be produced. The improved result of this process is that the thus-treated gelatin sets-up as a gel at the effective gelatin concentration of about 15-19 weight percent, as opposed to 20-45 weight percent without such treatment. This distinction is clearly evident from the differences between the kinematic viscosity of gelatin produced as described above, with and without thermal treatment of the lyophilized material. The viscosity is higher and set temperature lower for the thermally treated material for the same concentration of gelatin when subjected to the lyophilization (or other form of drying and moisture reduction) and thermal cross-linking, than when such treatment is not conducted.

In a further embodiment of this invention, a commercially available grade of gelatin, such as porcine gelatin, is utilized in the manufacture of the Bone Paste. Commercial grades of porcine gelatin are well known in the production of medicinal capsules and the like, and have also been used to produce a product known as GELFOAM, an insoluble matrix used in hemostatic applications. In this aspect of this embodiment, high-quality, commercially-available porcine gelatin, such as, for example, 250 to 300, including about 275 bloom commercial grade porcine gelatin available from DynaGel, Inc., (Plummer St. & Wentworth Ave., Calumet City, Ill. 60409), Vyse Gelatin Company, (5010 North Rose Street, Schiller Park, Ill. 610176), or the like, is purchased, packaged in an appropriately resistant packaging or double packaging, and is then sterilized by exposure to an appropriate dose of sterilizing radiation. For example, exposure to between about 2-3 MRad of Co⁶⁰, or an equivalently sterilizing dose of radiative exposure from another gamma radiation source. Following gamma irradiation, the gelatin is autoclaved under standard autoclave conditions known in the art. Subsequent to these treatments, the packaging of the sterilized gelatin is checked for integrity.

Prior to further processing, the sterilized gelatin is removed from the sterile packaging, and is ground to a particle size of about 1.5 mm or less, in a sterile grinder or blender. Subsequent to such grinding, the gelatin particles are intimately mixed with demineralized bone matrix (DBM), and the resistance of the blend to dissolution in an aqueous solvent at a given desired set-temperature is confirmed. Thus, for example, if the mixture is to be implanted in a human, resistance of the blend to dissolution in water or saline heated to approximately thirty-eight degrees centigrade is confirmed. If dissolution occurs, more gelatin is added to the formulation to ensure that the mixture is a solid at the desired set-temperature. According to this aspect of the invention, it is preferred for the DBM to be present at between about 1% to about 37%, and preferably about 24-33%, unless bone morphogenetic proteins (BMP's) or other osteogenic growth factors are present in the mixture, in which case the DBM concentration may be lowered. The gelatin is preferably present at between about 12-30%, depending on the desired set-temperature.

Subsequent to blending with DBM, if present, the composition is mixed with a sterile calcium-phosphate composition, such as hydroxyapatite, or more preferably, with sterilized cortico-cancellous bone chips. Since corticocancellous chips are only about 30-40% solid material, with the remainder of the volume thereof being void space, on a volume basis, a composition comprising about 100% (v/v) corticocancellous chips, with the 60-75% (v/v) of the composition being comprised of the gelatin/DBM or gelatin/BMP or other growth-factor blend, to form the final thermally sterilized bone paste composition of this invention.

Subsequent to blending, the radiatively and thermally sterilized bone paste composition described above is subjected to a further consistency check, to ensure resistance to dissolution of the bone paste composition in aqueous solution at a temperature at or below the desired set-temperature. The criticality of this aspect of the invention being that upon implantation, if the composition were to immediately liquify at physiological temperature, the effectiveness of the composition in inducing osteogenesis is reduced by too-rapid dissolution of the composition. In the event that the final composition does not remain solid at the desired set-temperature, increased concentrations of the gelatin are added to ensure solid setup of the composition at the desired temperature.

According to the method of producing the composition of this invention, the gelatin may be derived from the same or different species than that into which the composition is to be implanted. For example, human, porcine, bovine, fish, equine, feline, or canine gelatin is derived from collagen sources such as bone, skin, tendons, or cartilage, and may then be mixed with DBM or other osteogenic (osteoinductive or osteoconductive) materials. As noted above, the collagen is converted to gelatin via, liming, acidification or by enzymatic extraction, for example by pepsin or like enzymatic treatment, followed by denaturation by heat or other means. The gelatin may be derived from tissue by mastication of the tissue, followed by an extended treatment capable of breaking cross-links in the long collagen chains. In one embodiment, the tissue is ground then soaked for about 24-72 hours at between about 2-40° C. in dilute acid, such as 0.1 normal acetic acid. Preferably, an enzyme such as pepsin at a sufficiently high concentration is added. Pepsin concentrations of between about 10-20,000 i.u./liter, 100-2,000 i.u/liter, or like concentrations are added to the dilute acid at the start of the treatment, with the period of treatment being adjusted according to the enzyme concentration used. Solids are removed from the composition, for example by centrifugation, and the supernatant material in solution having a molecular weight of about 50,000 daltons or higher is retained. This may be achieved by any of a number of methods known in the art including, but not limited to, dialyzing the supernatant in a 50,000 dalton molecular weight cut-off membrane against several changes of solution, ultrafiltration against a membrane having a like molecular weight cut-off, (WCO) or gel permeation chromatography through a medium having a 50,000 dalton molecular mass cut-off. It will be recognized by those skilled in the art that the higher the MWCO of the gelatin, the lower the yield. Accordingly, lower MWCO gelatin preparations, down to abut 1000 dalton MWCO's could be used, recognizing that undesirable low molecular weight species might thereby be retained. Once again, the inclusion of the lyophilization and thermal cross linking disclosed above increases the yield of higher-molecular weight material.

In one embodiment of the invention, the gelatin solution resulting from the foregoing extraction is denatured, for example by heat-treatment to above about 50 to 650C. The denatured protein is then dried and subjected to an inert-gas, thermal cross-linking step described above. Thereafter, the gelatin may be stored in a dry state, or reconstituted with physiologically acceptable solutions and stored in a frozen state or it may be freeze-dried after reconstitution or it may be precipitated, for example in a volatile organic solvent, and reconstituted in a solution, such as an isotonic saline solution, at a concentration of between about 15-19% (w/w) gelatin.

The demineralized bone is preferably in a powdered form, and is preferably composed of particles in the size range between about 80-850 μm in diameter. Methods for producing demineralized bone powder are known in the art (see for example U.S. Pat. No. 5,405,390, herein incorporated by reference for this purpose), and are not, therefore, elaborated here. Demineralized bone powder, extracted by standard techniques, is mixed with the gelatin solution prepared as described above, to form a composition comprising about 1-40% (w/w) demineralized bone powder. Where present, bone morphogenetic proteins (BMP) reduce the percentage of DBM required in the composition. The BMP is preferably present at a concentration of between about 0.0001 to 10 mg/ml, 0.001 mg/ml to 4 mg/ml, or like concentration, depending on the amount of DBM present (1-40% w/w). In certain embodiments of this invention, and for particular orthopaedic applications in which strength of the bond formed by the thermally sterilized bone paste is important, addition of a bioactive glass is preferred. When added, the bioactive glass lowers the adhesiveness of the composition, but increases the stiffness of the composition upon setting. Accordingly, a bioactive glass, such as BIOGLASS® having a diameter of between about 0.5-710 μm, is added to the gel/demineralized bone composition. In addition, a composition comprising between about 0-40% (w/w) of bioactive glass with the gelatin forming about 11-19% (w/w) of the composition is also contemplated.

Compositions prepared as described above are easily extruded from a syringe, particularly when the temperature is elevated to above about 40° C., for example by immersion in a water bath, by limited treatment in a microwave, by placement in a syringe warmer, or any of a number of other methods for heating the container. The extruded gel is resilient, sticky and easily formable into any desired shape. In addition, the composition retains its strength and is poorly soluble in saline or water once it sets-up.

Accordingly, having generally described the composition of this invention, and taking into account the specifics of the exemplary support provided below, the following guidelines for the preparation and use of the composition of this invention are provided:

The gelatin from DBM should be prepared at a temperature between about 30 and 37° C. While the yield is higher (60%) at 37° C., the quality, based on measured kinematic viscosity, is slightly lower than that produced at 30° C. Preferably, the gelatin is produced by limited exposure of collagen to an enzyme, such as pepsin, or like enzyme. A concentration of pepsin set at 300 U/L-500 U/L works well, but those skilled in the art will recognize that a wide range of enzyme concentrations could be tested, based on what is disclosed herein. Those skilled in the art will recognize that acid or alkaline processing of skin and tendon may be an alternative to the pepsin technique.

The final composition preferably comprises gelatin solution having a viscosity of about 3600 centipoise or higher at 44° C. (when measured in the linear range of a viscosity/sheer rate plot−0.87/s), or a kinematic viscosity of about 0.7 centistokes at 44° C. The concentration of the gelatin in the carrier phase (i.e. absent added osteogenic components) is preferably about 15-19% (w/w), to ensure that gellation at 38° C. will occur in a reasonable amount of time. Naturally, those skilled in the art will recognize that, depending on the species of the organism into which the composition is to be implanted, different temperatures may be required. These needs are accommodated by altering the gelatin concentration, increasing the concentration if a higher gel temperature is desired, and lowering the concentration if a lower gel temperature is desired.

The DBM content of the composition is defined herein by the concentration required to obtain bone formation similar to that seen with DBM alone. We have found that about 5-40% (w/w) DBM in the composition is effective. Anything lower than about 5% seems to do very little by way of bone formation, unless added BMPs (component iii) are present in the composition, in which case the DBM concentration may be substantially reduced or eliminated altogether. Naturally, based on this disclosure, those skilled in the art will recognize that by addition of different concentrations and compositions of bone morphogenetic proteins or other osteogenic or osteoinductive factors, the weight percent of DBM in the composition may be manipulated up or down. In addition, it will be recognized that, depending on the species into which the composition is implanted, the DBM weight percent may need to be adjusted up or down.

We have found in in vivo studies that the compositions with DBM contents from 15 to 33% all produce calcified tissue. We have found that there is a good correlation between the amount of DBM in the composition and the level of bone induction, as long as the DBM concentration is greater than about 19% (w/w). About 38-40% (w/w) is the upper mass limit for DBM. Accordingly, 1-40% (w/w) DBM, and more preferably 5-30% (w/w), 7-33% (w/w) or 15-25% (w/w) is desirable for this component.

We have observed histologically that, subsequent to implantation into an animal, the gelatin phase is totally absorbed within about 2 weeks. Additionally, cartilage and mineralized bone formed within two weeks, with mature bone being evident by about the fourth week. The animals in these studies did not exhibit any gross health problems or any indications of irritation, hematoma, soreness, fever, or weight loss during the study.

The composition according to this invention, whether it comprises gelatin and osteogenic components (i-iv) may act as a carrier for cortical, cancellous or cortical and cancellous bone chips. Such compositions are useful for filling larger bone voids. In addition, when these bone chips are not demineralized, they provide an added spectrum of biological properties not exhibited by the gelatin alone or the gelatin plus osteogenic components (i-iv). When present, it is preferred for such bone chips to be in the size range of about 80 μm to about 10 mm.

In a further embodiment of this invention, the composition of gelatin and osteogenic components (i-iv) is injection molded, vacuum molded, rotation molded, blow molded, extruded or otherwise formed into a solid form. Such forms would desirably take the form of vertebral disks, acetabular hemispheres, formable inserts for repairing acetabular cup defects, tubes, ellipsoid shapes for void filling, and intramedullary plugs, which are useful to plug the intramedullary canal of various bones (i.e. the marrow containing portion of the bone) to prevent bone cement from entering healthy bone tissue. These forms are produced, for example, by raising the temperature of the composition above its liquefaction temperature (e.g. about 45° C.), and allowing the composition to gel in a mold of appropriate shape. For such forms, the gelatin content is preferably made as high as possible to ensure that the form remains solid upon grafting into a vertebrate recipient.

Having described the composition of this invention and the method of its preparation, the manner of using the composition is next set forth. Methods of use of the composition include extrusion, via injection, or molding, either by hand or mechanically, to produce appropriately shaped implants, either in vivo or ex vivo, with subsequent implantation into a desired implantation site. The composition may be directly applied to the site of non-union fractures, injected between vertebrae that are to be fused, molded into any physiologic shape desired and applied in any orthopedic context in which osteoconduction, or osteoinduction is desired. The composition may likewise be used to coal allograft, autograft, xenograft, metallic, synthetic bioabsorbable or any other type of implant to enhance the osseointegration of the implant and osteoconduction and osteoinduction around the implant. This use of the bone paste composition of this invention is particularly useful for porous implants.

Having generally described the invention, the following examples are provided to show specific features and applications of the invention. It should be recognized that this invention is in no way limited to the specifics of the examples as set forth below, and that the limits of this invention are defined by the claims which are appended hereto.

### EXAMPLE 1

### Procedure for Definition of the Set Temperature of the Thermally Sterilized Bone Paste

In this procedure, the set temperature of the thermally sterilized bone paste is defined. An aliquot of the matrix stored in a frozen state was thawed at approximately 45° C. and then drawn into a syringe. The aliquot was then syringed into a tube which is equilibrated at 38° C. for 15 minutes. Another aliquot was syringed into a vial of distilled water equilibrated at 380° C. and allowed to sit for 15 minutes. After this period of time, both aliquots of the matrix were solid and there was little or no dissolution of the matrix into the distilled water.

### EXAMPLE 2

### Method for Defining the Concentration of Gelatin in the Thermally Sterilized Bone Paste

For each composition, a matrix is established as follows:

| Gelatin(g) | DBM(g) | Water(g) |
|---|---|---|
| 0.15 | 0.33 | 0.52 |
| 0.17 | 0.33 | 0.50 |
| 0.19 | 0.33 | 0.48 |

These masses, in a powder form were placed in a plastic bag or like malleable but liquid impermeable container prior to addition of the indicated mass of water. The bag was sealed and shaken to mix the powders thoroughly. The water was added and the bag resealed. The ingredients were kneaded while the bag was submerged in a water bath set at approximately 42-47° C., and then the composition was tested for set-up at 38° C. as described in Example 1.

### EXAMPLE 3

### Comparison of the Osteoinductivity of Grafton® (DBM in glycerol, Osteotech), DBM, and Thermally Sterilized Bone Paste with Suspended DBM in an Athymic Rat Intramuscular Model

Known masses of commercially available Grafton® (DBM in glycerol, Osteotech), DBM, powdered DBM, and the Thermally sterilized bone paste of this invention in which DBM was suspended were implanted intramuscularly into athymic nude rats according to the model of Strates and Urist (Urist, Clin. Ortho. Rel. Res. 71:271-278, 1970). The osteoinductivity of the DBM included in the thermally sterilized bone paste of this invention and that included in Grafton® were found to be identical in a standard osteosarcoma induction assay. After 21 days, explants were removed and analyzed by X-ray and atomic absorption. This analysis revealed a calcium deposition yield of: 0.40±0.17 g/ml implant for the thermally sterilized bone paste/DBM composition; 0.039±0.094 g/ml for Grafton®; and 0.15±0.072 g/ml for DBM alone. Therefore, DBM in the composition of this invention yielded a 2.7 fold increase in bone induction than DBM alone, and 10.3 fold the bone induction attributable to Grafton®. All of these differences were statistically significant. In addition, the osteoinductive effect of Grafton® was variable, forming bone in only six of ten implants. The DBM/thermally sterilized bone paste of this invention induced bone formation in all implants.

### EXAMPLE 4

### Thermally Sterilized Bone Paste Production. Kinematic Viscosity, and Critical Concentration for Gellation at 38° C.

The kinematic viscosity of porcine gelatin subjected to different heat sterilization treatments (121° C., 6 hours; 130° C., 3 hours; 150° C., 2.5 hours) was compared to the kinematic viscosity of porcine gelatin which had not been subjected to heat sterilization. The lyophilized gelatin samples treated for 3 hours at 130° C. required approximately 40-50 minutes to dissolve in sterile water at 550° C., while the lyophilized gelatin samples treated for 6 hours at 121° C. required only about 10 minutes to dissolve in sterile water. The porcine material treated for 2.5 hours at 150° C. was not soluble. We found that the kinematic viscosity of the thermally treated porcine samples (121° C. and 130° C.) increased, as compared with that of the untreated porcine samples. A similar study of human material (121° C., 6 hours), substantially reproduced this result.

By way of background, the thermally sterilized bone paste was produced from gelatin extracted from demineralized human cortical bone powder in the size range of 250-850 μm, also referred to as demineralized bone matrix powder (DBM), by treatment with 0.5 M. acetic acid, and pepsin. The DBM was incubated for from 5 to 24 hours at 30° C. The supernatant was retained and the solid material was treated with a fresh solution of acetic acid/pepsin at 33° C. for another 5 to 24 hours. The supernatants were combined, and the pH was adjusted to 7.0 with 1 N NaOH, deactivating the pepsin. The solution was pumped at a controlled rate through a tube submerged in a 60° C. water bath such that all portions of the solution were subjected to the 60° C. treatment for a full 15 minutes, then quenched in ice water. The solution was centrifuged and the supernatant was either dialyzed or diafiltered against a 30,000 daltons molecular weight cut off membrane. The retentate was then lyophilized. As a control, dry samples were retained without further heat treatment. The remainder of the material was autoclaved in sealed vials that were first evacuated to 100 millitorr and then back-filled with nitrogen, and sealed. The autoclaving was continued for either six hours at 121° C., or the samples were treated for three hours at 130° C.

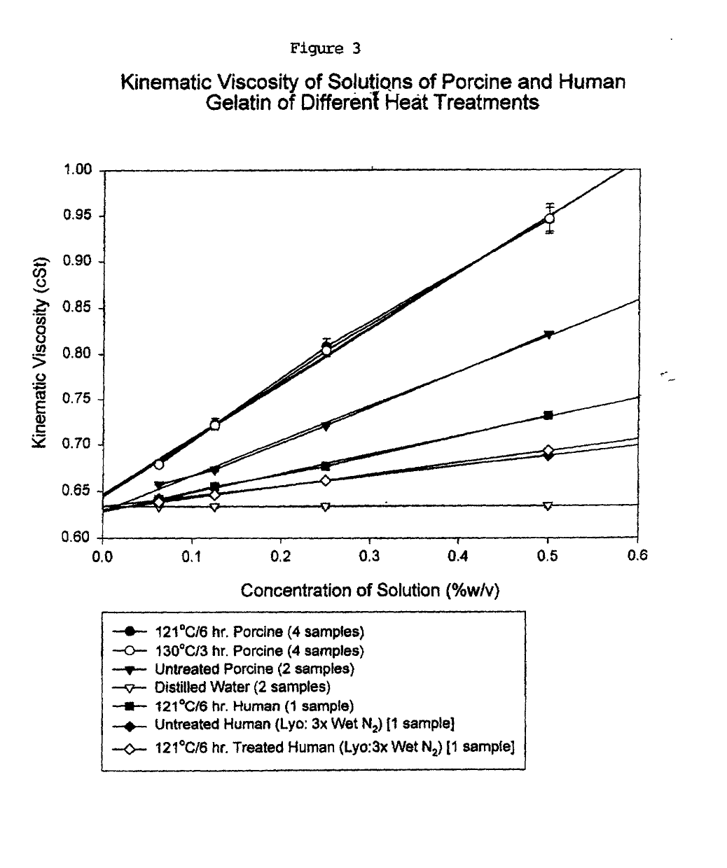

In FIG. 3, the results of heat treating four samples of porcine material at 121° C. for 6 hours (solid circles), four samples of porcine material at 130° C. for 3 hours (open circles), is compared with two samples of untreated porcine material (solid triangles). In addition, one sample of human material treated for 6 hours at 121° C. (closed square) is compared with one sample of untreated human material (closed diamond), and a sample of human material treated for six hours at 121° C., but under an atmosphere of moist (about 1% humidity) nitrogen (open diamond).

The kinematic viscosities of dilute concentrations of the thus treated materials (0.5%, 0.25%, 0.125%, 0.0625% in phosphate buffered saline solutions (pH 7.4 at 25° C.), were measured with an Ubbelhode viscometer at 44° C. The kinematic viscosities (centistokes) were graphed versus concentration, FIG. 3. The linear regression was extrapolated to zero to determine the kinematic viscosity at zero concentration.

To determine the set temperatures for various thermally sterilized bone paste compositions, gelatin concentrations were varied from 15 w/w % of total composite to 19 w/w % of total composite in water. All thermally sterilized bone paste composites tested contained DBM at a concentration of 33 w/w % of the total composite. Different ambient temperatures were used to test whether the thermally sterilized bone paste was solid or liquid, 45° C., 43° C., 41° C., 40° C., 38° C., and 35.5° C. The set temperature was determined both by subsequent lowering of the ambient temperature and raising of the ambient temperature. The critical concentration of gelatin in a thermally sterilized bone paste composite that was solid at slightly above human body temperature, 38° C. to 39° C., was 15 w/w % −19 w/w % of the total composite for human gelatin, processed at 33° C., and with 33 w/w % of the composite being DBM, the remainder being PBS or water. The human gelatin processed at 33° C., had a zero concentration kinematic viscosity of 0.65 centistokes. Human gelatin solutions of lower kinematic viscosities were found to have critical concentrations in excess of about 19 w/w %. Correspondingly, gelatins with viscosities higher than about 0.65 centistokes are expected to thermally cross-link at concentrations lower than about 15% (w/w).

### EXAMPLE 5

### Procedure for the Production of the Thermally sterilized bone paste of this Invention

This example provides one procedure for the manufacture of bone paste from gelatin and demineralized bone. As fractions of the total mass of composition desired, the following components are weighed (percentages given are of total composite weight):

| | |
|---|---|
| Dry demineralized bone: | 1-40% (w/w) |
| Lyophilized thermally | 11-30% (w/w) |
| cross-linkable gelatin: | |
| BIOGLASS ®: | 0-40% (w/w) |
| bone morphogenetic protein: | 0-10 mg/ml |

These components are thoroughly blended while dry, and the balance of the composition mass is made up by addition of water, phosphate buffered saline, or any other physiologically acceptable liquid carrier. The composition may be packaged in this form or lyophilized for later reconstruction with water. The malleable properties of the An, composition are achieved by heating the composition to a temperature sufficient to exceed the liquefaction point of the gelatin, and then allowing the composition to cool to the temperature at which it gels.

### EXAMPLE 6

### Procedure for Producing a Radiatively and Thermally Sterilized Bone Paste Composition of this Invention

According to this aspect of the invention, a commercially available grade of porcine gelatin was utilized in the manufacture of the Bone Paste. Porcine gelatin, 275 bloom commercial grade from DynaGel, Inc., (Plummer St. & Wentworth Ave., Calumet City, Ill. 60409), was purchased, packaged in a sealed double packaging, and sterilized by exposure to 2-3 MRad of Co⁶⁰. Following gamma irradiation, the gelatin was autoclaved under standard autoclave conditions (20 minutes, 121 degrees). Subsequent to these treatments, the packaging of the sterilized gelatin was checked for integrity.

Prior to further processing, the sterilized gelatin was removed from the sterile packaging, and was ground to a particle size of about 1.5 mm or less, in a sterile grinder or blender. Subsequent to such grinding, the gelatin particles were intimately mixed with demineralized bone matrix (DBM) in a sterile sausage-grinder, and the resistance of the blend to dissolution in an aqueous solvent at 38 degrees centigrade. Compositions containing DBM at concentrations of about 1% to about 37%, including concentrations of about 24-33%, were prepared in this manner. In compositions in which bone morphogenetic proteins (BMP's) or other osteogenic growth factors were added the DBM concentration was reduced to as low as 0%. The gelatin was included in the composition at concentrations between about 12-30%, depending on the desired set-temperature.

Subsequent to blending with DBM, if present, the composition was mixed with a sterile calcium-phosphate composition, such as hydroxyapatite, or with sterilized cortico-cancellous bone chips. Since corticocancellous chips are only about 30-40% solid material, with the remainder of the volume thereof being void space, on a volume basis, a compositions were prepared comprising about 100% (v/v) corticocancellous chips, with the 60-75% (v/v) of the composition being comprised of the gelatin/DBM or gelatin/BMP or other growth-factor blend, to form the final thermally and irradiatively sterilized bone paste composition of this invention.

Subsequent to blending, the radiatively and thermally sterilized bone paste composition described above was subjected to a further consistency checks, to ensure resistance to dissolution of the bone paste composition in aqueous solutions at a temperature at or below the desired set-temperature.

As a result of the foregoing processing, a composition having the following characteristics is produced: a melt-flow-index (MFI) from a one cubic-centimeter BD (Beckton-Dickinson), slip-tip syringe of greater than about 0.00719 g/sec, or greater than about 0.03497 g/sec from a five cubic centimeter BD slip-tip syringe at 47±2 degrees centigrade when 2644±1 gram of weight is applied to a plunger of a syringe containing said composition. A further characteristic of such formulations is that it does not dissolve within 5 minutes when placed in distilled water at 38±0.5 degrees centigrade.

Implantation of these compositions are anticipated to result in induction of bone formation at the site of implantation.

### References

Cornell, C. Techniques in Orthopaedics 1992, 7, 55-63.

Bloebaum, R. D. Human Bone Ingrowth and Materials; Bloebaum, R. D., Ed.; Society for Biomaterials: Denver, CO, 1996.

Einhorn, T. A. Enhancement of Bone Repair Using Biomaterials; Einhorn, T. A., Ed.; Society for Biomaterials: Denver, CO, 1996.

Benedict, J. J. The Role of Carrier Matrices on Bone Induction In Vivo; Benedict, J. J., Ed.; Society for Biomaterials: Denver, CO, 1996.

Strates, B.; Tiedeman, J. European Journal of Experimental Musculoskeletal Research 1993, 2, 61-67.

Urist, M. R. Bone Morphogenetic Protein; Urist, M. R., Ed.; W. B. Saunders Co.: Philadelphia, 1992, pp 70-83.

Yazdi, M.; Bernick, S.; Paule, W.; Nimni, M. Clinical Orthopaedics and Related Research 1991, 262, 281-285.

Younger, E.; Chapman, M. Journal of Orthopaedic Trauma 1989, 3, 192-195.

Hardin, C. K. Otolaringologic Clinics of North America 1994, 27, 911-925.

Senn, N. The American Journal of the Medical Sciences 1889, 98, 219-243.

Urist, M. R.; Huo, Y. K.; Brownell, A. G.; Hohl, W. M.; Buyske, J.; Lietze, A.; Tempst, P.; Hunkapiller, M.; DeLange, R. J. Procedures of the National Acadamy of Sciences, USA 1984, 81, 371-375.

Urist, M. R.; Chang, J. J.; Lietze, A.; Huo, Y. K.; Brownell, A. G.; DeLang, R. J. Methods in Enzymology 1987, 146, 294-313.

Lasa, C.; Hollinger, J.; Droham, W.; MacPhee, M. Plastic and Reconstructive Surgery 1995, 96, 1409-1417.

Nathan, R.; Bentz, H.; Armstrong, R.; Piez, K.; Smestad, T.; Ellingsworth, L.; McPherson, J.; Seyedin, S. Journal of Orthopaedic Research 1988, 6, 324-334.

Hench, L. L.; Andersson, O. H. Bioactive Glasses; Hench, L. L.; Andersson, O. H., Ed.; World Scientific Publishing Co. Pte. Ltd.: Singapore, 1993, pp 41-63.

Scarborough, N. Bone Repair Using Allografts; Scarborough, N., Ed.; Society for Biomaterials, 1996.

Frenkel, S. R.; Moskovich, R.; Spivak, J.; Zhang, Z. H.; Prewett, A. B. Spine 1993, 18, 1634-1639.

Sperling, L. H. Introduction to Physical Polymer Science; John Wiley and Sons, Inc.: New York, 1992.

McDonald, T. O.; Britton, B.; Borgmann, A. R.; Robb, C. A. Toxicology 1977, 7, 37-44.

Culling, C. F. A.; Allison, R. T.; Barr, W. T. Cellular Pathology Technique; 4 ed.; Butterworths: London, 1985.

U.S. Pat. No. 5,481,601

U.S. Pat. No. 5,236,456

U.S. Pat. No. 5,405,390

U.S. Pat. No. 4,440,750

U.S. Pat. No. 4,394,370

U.S. Pat. No. 4,472,840

U.S. Pat. No. 4,678,470

WO 89/04646

1. An implantable bone paste composition comprising thermally sterilized gelatin as a carrier for substantially bioabsorbable osteogenic components for use in a recipient in need thereof.

2. The bone paste composition of claim 1 when implanted in non-union fractures, periodontal ridge augmentation, craniofacial surgery, arthrodesis of spinal or other joints, spinal fusion procedures, and implant fixation.

3. The composition of claim 1 wherein the gelatin is thermally cross-linkable at or slightly above the temperature of the organism into which it is to be implanted.

4. The composition of claim 3 wherein said composition gels at about 38° C.

5. The composition of claim 3 wherein said gelatin is present at a concentration of between about 11-19% (w/w) gelatin as a fraction of the weight of the composition.

6. The composition of claim 5 wherein the osteogenic component is selected from the group consisting of:
(i) demineralized bone, preferably derived from the species into which the thermally sterilized bone paste is to be implanted; or
(ii) bioactive glass ceramic, BIOGLASS®, bioactive ceramic, calcium phosphate ceramic, hydroxyapatite, hydroxyapatite carbonate, corraline hydroxyapatite, calcined bone, cortical bone chips, cancellous bone chips, tricalcium phosphate, like material, or mixtures thereof; or
(iii) bone morphogenetic protein, osteogenic proteins or peptides and the like, TGF-beta, bone marrow extracts, vascular proliferation or regeneration growth factors, PDGF, or mixtures thereof, natural or recombinant; or
(iv) mixtures of (i)-(iii).

7. The composition of claim 6 wherein the gelatin, the demineralized bone matrix, or both are derived from the species into which the bone paste is to be implanted.

8. The composition of claim 7 wherein DBM is present at between about 1-40% (w/w) of the total composite weight.

9. The composition of claim 8 wherein DBM is present at between about 15-33% (w/w) of the total composite weight.

10. The composition of claim 6 wherein the bioactive glass is BIOGLASS®.

11. The composition of claim 6 wherein component (ii) is present at between about 0-60% (w/w) of the total composition mass.

12. The composition of claim 6 comprising antibiotics, bone morphogenetic or other proteins, whether derived from natural or recombinant sources, wetting agents, glycerol, carboxymethyl cellulose (CMC), growth factors, steroids, non-steroidal anti-inflammatory compounds, or combinations thereof.

13. The composition of claim 6 comprising between about 0.0001 to 10 mg/ml bone morphogenetic protein, natural or recombinant.

14. The composition of claim 1 which is a frozen solution or is freeze-dried.

15. The composition of claim 1 wherein the gelatin is human, bovine, porcine, ovine, fish, equine, feline, canine or mixtures thereof.

16. The composition of claim 1 wherein the gelatin is derived from human collagen sources via enzymatic, acid or alkaline extraction.

17. The composition of claim 16 wherein said human collagen sources are human skin, bone, cartilage, tendon, connective tissue, or mixtures thereof.

18. The composition of claim 17 produced by:
(a) treating the collagen source with pepsin at about 30° C., separating a soluble supernatant from an insoluble residue, and retaining the soluble supernatant;
(b) treating the insoluble residue with pepsin at about 330° C., separating a soluble supernatant from an insoluble residue, and retaining the soluble supernatant;
(c) pooling the thus obtained soluble supernatants;
(d) heat denaturing the pooled supernatants under controlled conditions to produce gelatin;
(e) removing the moisture from the gelatin to produce dry gelatin;
(f) thermally sterilizing the dry gelatin; and
(g) mixing a known mass of the dry gelatin with a known mass of osteogenic compound such that the dry gelatin is present at a final concentration of about 11-19% (w/w).

19. The composition of claim 18 wherein the denaturation is achieved by heating to at least 60° C.

20. The composition of claim 19 wherein the gelatin has a molecular weight of greater than about 50,000 daltons.

21. The composition of claim 20 wherein the step of thermally sterilizing the dry gelatin occurs at between about 121° C. to 130° C. for between about five minutes and 18 hours.

22. The composition of claim 1 wherein the osteogenic component is demineralized bone matrix in a powdered form, and is composed of particles in the size range between about 80-850 μm in diameter.

23. The composition of claim 22 comprising about 1-40% (w/w) demineralized bone matrix powder, provided that if the demineralized bone matrix powder is absent, then a bone growth factor is present at a concentration of at least 0.0001 mg/ml.

24. The composition of claim 23 wherein said bone growth factor is morphogenetic protein, TGF-β, osteoinductive factors, osteoconductive factors, or mixtures thereof, natural or recombinant.

25. The composition of claim 6 wherein the bioactive glass is BIOGLASS® having a diameter of between about 0.5-710 μm.

26. The composition of claim 1 further comprising cortical, cancellous or cortical and cancellous bone chips.

27. The composition of claim 26 wherein said bone chips are in the size range of 80 μm to 10 mm.

28. The composition of claim 1 which is injection molded, vacuum molded, rotation molded, blow molded, extruded or otherwise formed into a solid form.

29. The composition of claim 28 wherein said form is selected from vertebral disks, acetabular hemispheres, tubes, ellipsoid, oblong, and “U” shapes for void filling, intramedullary plug formation, and impaction grafting.

30. A method for inducing bone formation in vivo in a recipient in need thereof which comprises implanting an effective amount of an implantable bone paste composition comprising thermally sterilized gelatin as a carrier for substantially bioabsorbable osteogenic components.

31. The method claim 30 which comprises repairing non-union fractures, achieving periodontal ridge augmentation, conducting craniofacial surgery, securing implants, arthrodesis of spinal or other joints, spinal fusion procedures, or impaction grafting, which comprises implanting said composition at the site in vivo in need of such treatment.

32. The method according to claim 31 which comprises extruding said composition from a syringe at a first temperature at which it remains liquid or highly malleable, and forming a resilient, sticky and easily formable shape from said composition as it gels at a second temperature at or slightly above the body temperature of the organism into which it is implanted.

33. A method for making an implantable graft which comprises preparing thermally sterilized composition comprising a thermally cross-linkable gelatin carrier and suspending therein a substantially bioabsorbable osteogenic component.

34. The method of claim 33 wherein said osteogenic component is selected from:
(i) demineralized bone, preferably derived from the species into which the thermally sterilized bone paste is to be implanted; or
(ii) bioactive glass ceramic, BIOGLASS®, bioactive ceramic, calcium phosphate ceramic, hydroxyapatite, hydroxyapatite carbonate, corraline hydroxyapatite, calcined bone, cortical bone chips, cancellous bone chips, tricalcium phosphate, like material, or mixtures thereof; or
(iii) bone morphogenetic protein, osteogenic proteins or peptides and the like, TGF-beta, bone marrow extracts, vascular proliferation or regeneration growth factors, PDGF, or mixtures thereof, natural or recombinant; or
(iv) mixtures of (i)-(iii).

35. The method of claim 34 which further comprises injection molding, vacuum molding, rotation molding, blow molding, extruding or otherwise forming said composition into the desired form of a solid graft, and allowing the composition to solidify at a temperature at which the gelatin becomes thermally cross-linked.

36. The method of claim 35 wherein said form is selected from vertebral disks, acetabular hemispheres, tubes, ellipsoid, oblong, and “U” shapes for void filling, intramedullary plug formation, and impaction grafting.

37. The method of claim 35 which comprises raising the temperature of the composition above its liquefaction temperature and allowing the composition to gel in a mold of appropriate shape.

38. The method of claim 33 wherein the composition is thermally sterilized by treatment of the dry gelatin at between about 121° C. to 130° C. for between about 5 minutes to about 18 hours, prior to suspending therein a substantially bioabsorbable osteogenic component.

39. The bone paste composition of claim 1 wherein said gelatin is sterilized by exposure to a sterilizing dose of gamma irradiation.

40. The composition according to claim 39 comprising, on a volume basis, 60-75% of component (a), and between 0-100% on a volume basis, of component (b), which, if present, because of the substantial void volume thereof, completely absorbs any volume contribution of component (a), wherein component (a) comprises:
about 11-30% (w/w) gelatin, 24-33% (w/w) demineralized bone matrix, with the balance being made of water or an aqueous solution;
and wherein component (b) comprises aseptic corticocancellous bone chips.

41. The composition according to claim 40 wherein component (a) comprises between about 15-19% gelatin.

42. The method according to claim 33 comprising exposing said gelatin to a sterilizing dose of gamma irradiation.

43. The method according to claim 42 wherein said gelatin is thermally sterilized by autoclaving the gelatin.

44. The method according to claim 43 wherein said gelatin is a commercially available grade of porcine gelatin.

45. The method according to claim 44 wherein said gelatin has a bloom number of between about 250 and 300.

46. The composition according to claim I having a melt-flow-index (MFI) from a one cubic-centimeter BD slip-tip syringe of greater than about 0.00719 g/sec, or greater than about 0.03497 g/sec from a five cubic centimeter BD slip-tip syringe at 47±2 degrees centigrade when 2644±1 gram of weight is applied to a plunger of a syringe containing said composition.

47. The composition according to claim 1 wherein said composition does not dissolve within 5 minutes when placed in distilled water at 38±0.5 degrees centigrade.
